(19) United States
(12) Patent Application Publication
(10) Pub. No.: US 2009/0075231 A1
(43) Pub. Date: Mar. 19, 2009

(54) DENTAL/MEDICAL ORAL IRRIGATION SYSTEM

(76) Inventors: Russell C. Tontz, San Diego, CA (US); Robert S. White, Rolling Hills Estates, CA (US)

Correspondence Address: NYDEGGER & ASSOCIATES, 348 OLIVE STREET, SAN DIEGO, CA, 92103, US

(21) Appl. No.: 11/855,434

(22) Filed: Sep. 14, 2007

(51) Int. Cl. A61M 3/00

(52) U.S. Cl. 433/88; 604/518

(57) ABSTRACT

A method and device delivers a medicament to a selected tissue for treatment of the tissue, plaque and biofilm included. In the device, a first reservoir member is used to connect to a fluid source. Further, a second reservoir member engages the first reservoir member to define a chamber for receiving the medicament. Structurally, the second reservoir member includes an outlet. Attached to the outlet is a tube that extends to a nozzle. When connected, the first reservoir member, second reservoir member, and tube define a passageway from the fluid source to the nozzle. During use, a flow rate for a fluid is established through the passageway to deliver the medicament from the passageway through the nozzle to the selected tissue.

### FIELD OF THE INVENTION

The present invention pertains generally to medication delivery devices and to methods for using such devices. More particularly, the present invention pertains to medication delivery devices that introduce medication to a patient in a stream of fluid. The present invention is particularly, but not exclusively, useful as a fluid delivery device with an inline medication reservoir.

### BACKGROUND OF THE INVENTION

Dental diseases, including dental caries and periodontal disease, comprise one of the most common health disorders in man. Further, these dental diseases have been sources for other health disorders that have afflicted man throughout history. It is now well known, these diseases are highly associated with or caused by contagious micro-organisms that are transmitted by various methods. For instance, the transmission of contagious micro-organisms may be air-borne, food-borne, contact-borne, STD-borne, saliva-borne, or through other means.

Research assays indicate that when micro-organisms enter the body through portals of entry, including the mouth, nose and ears, they may then be transferred into the blood stream. Once in the blood stream, the micro-organisms can move to other systemic areas such as the heart, brain, lungs, stomach, nervous system, uterus, digestive tract, pancreas and other sites where they are able to cause a health disorder. Importantly, the health concerns associated with these disorders cannot be ignored. And, it happens, the mouth is a primary source of concern. At the present time, about forty harmful micro-organisms involved in dental caries and periodontal disease have been identified that can directly cause or contribute to health disorders in other parts of the body.

While it is known that anti-microbial agents will reduce and help eliminate the contagious micro-organisms in the mouth, there is not yet a completely effective system or method for the delivery of an anti-microbial agent. For instance, tooth brushing and dental flossing do not provide complete and proper delivery of anti-microbial agents to specific infected sites. Furthermore, infected sites harboring different specific species of micro-organisms must typically require the delivery of multiple anti-microbial agents.

In light of the above, it is an object of the present invention to provide a device for delivering anti-microbial agents to selected tissue sites. Another object of the present invention is to provide a medicament delivery device having an inline reservoir for holding a dissolvable anti-microbial agent. Still another object of the present invention is to provide a device for delivering selected anti-microbial agents to a selected tissue at predetermined concentrations and at predetermined pressures. It is another object of the present invention to provide a method and device for delivering medicaments to selected tissues that is easy to implement, cost effective and simple to use.

### SUMMARY OF THE INVENTION

In accordance with the present invention, a device is provided for delivering a medicament, specifically an anti-microbial agent, to a selected oral tissue. Structurally, the device includes a first reservoir member for fluid communication with a fluid source. Further, the device includes a second reservoir member that engages with the first reservoir member to define a chamber. Also, the second reservoir member includes an outlet. Connected to the outlet is a tube that extends to a nozzle. Together, the first and second reservoir members and the tube define a passageway from the fluid source to the nozzle. As constructed, the passageway passes through the chamber.

For purposes of the present invention, a medicament is positioned in the chamber. In order to prevent the medicament from blocking the outlet of the second reservoir member, the device is provided with a filter at the outlet. Further, the device is provided with a plurality of first and second reservoir members of differing sizes. As a result, different amounts of medicament may be positioned in the chamber formed by the selected reservoir members.

During operation of the device, the first reservoir member is connected to a fluid source. Also, the medicament is positioned between the first and second reservoir members, and the first and second reservoir members are then engaged with each other. After the tube is connected to the outlet of the second reservoir member, a flow rate for the fluid is established. In this manner, the fluid flows through the chamber, dissolving the medicament, and carrying the medicament through the nozzle to the selected tissue. The operation may be repeated for other desired anti-microbial agents, using different desired amounts and concentrations of the agents. As a result, the selected oral tissue is irrigated with multiple anti-microbial agents to reduce and eliminate targeted disease associated micro-organisms.

For instance, topical antiseptics may be used in an initial oral disinfecting process. If resistant infections are encountered, antibiotics may be used in a subsequent process(es) for one to two weeks, depending on the antibiotic, and its combination with other antibiotics (specificity). This method recognizes that viruses are essential cofactors in the periodontal disease process. Before, only bacteria, fungi and protozoans were implicated in these infections. Now, herpes, HPV (human papilloma virus), cytomeglia viruses, and other viruses are being investigated. The sole use of commercially available topical antibiotics as controlled release devices suffer from several potential problems, including insufficient spectrum of antimicrobial activity in some periodontal polymicrobial infections, risks of producing an antibiotic resistant microbiota, and high acquisition costs. The recommended treatment with antimicrobial agents, such as available chemotherapeutics, can provide effective, safe, practical and affordable means of controlling subgingival colonization of periodontal pathogens and various types of periodontal disease.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic view of a device for delivering a medicament to a selected tissue in accordance with the present invention; and

FIG. 2 is a cross sectional view of the reservoir members of FIG. 1.

### DESCRIPTION OF THE PREFERRED EMBODIMENTS

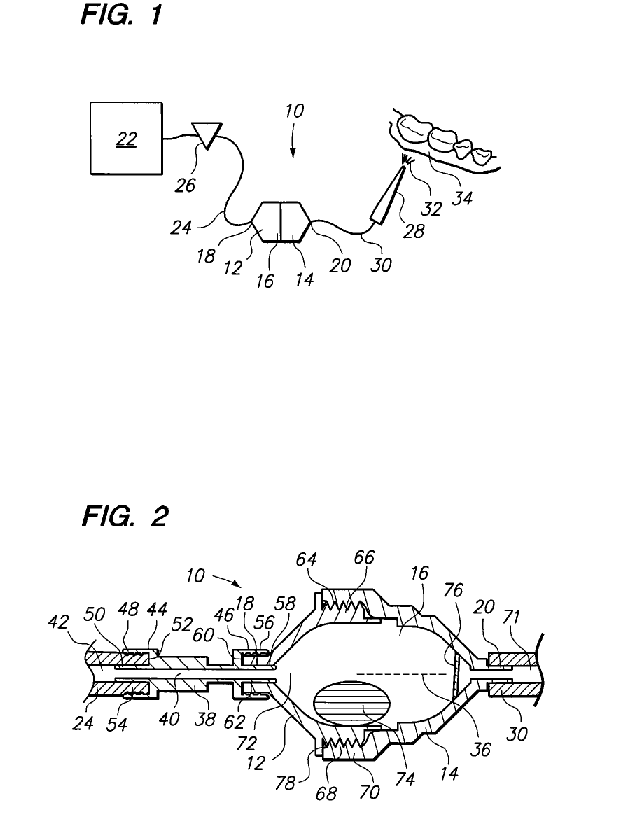

Referring initially to FIG. 1, a medicament delivery device is shown, and is generally designated 10. As shown, the device 10 includes a first reservoir member 12 interconnected with a second reservoir member 14. Structurally, the reservoir members 12, 14 define an inner chamber 16. Further, the first reservoir member 12 forms an inlet 18 to the chamber 16, while the second reservoir member 14 forms an outlet 20 from the chamber 16. As shown, the inlet 18 is connected to a fluid source 22 via a tube 24. Also, a valve 26 is positioned along the tube 24 to control the flow rate of fluid from the fluid source 22. In FIG. 1, it can be seen that the outlet 20 is connected to a nozzle 28 via a tube 30. As shown, the nozzle 28 may direct fluid 32 from the fluid source 22, through the chamber 16, to selected tissue 34, such as oral tissue, for medical treatment.

In FIG. 2, it can be seen that the chamber 16 defines a chamber axis 36. Further, the engagement between the tube 24, reservoir members 12, 14, and the tube 30 is illustrated more clearly. As shown, the device 10 includes an anchor member 38 defining a channel 40 interconnecting the inlet 18 of the first reservoir 12 with the lumen 42 of the tube 24. Structurally, the anchor member 38 extends axially from a proximal end 44 to a distal end 46. At its proximal end 44, the anchor member 38 forms a threaded bore 48 radially spaced from a protrusion 50 extending axially in the proximal direction. Between the threaded bore 48 and the protrusion 50, the anchor member 38 forms a cylindrical void 52. As shown in FIG. 2, the distal end 54 of the tube 24 is forced into the cylindrical void 52 to connect the lumen 42 of the tube 24 with the channel 40 of the anchor member 38. As shown, the tube 30 is flexible and is deformed as it engages the anchor member 38.

Still referring to FIG. 2, it can be seen that the distal end 46 of the anchor member 38 also forms a threaded bore 56 and protrusion 58 that define a cylindrical void 60. As shown, the inlet 18 of the first reservoir member 12 is received within the cylindrical void 60. In order to ensure a fluid tight engagement, the inlet 18 is provided with tabs 62 that extend radially outward to engage with the threaded bore 56. The first reservoir member 12 also includes radially outward extending threads 64 at its distal end 66. Further, the second reservoir member, 14 includes reciprocating, radially inward extending threads 68 at its proximal end 70. With this cooperation of structure, the reservoir members 12, 14 may be connected and disconnected. In FIG. 2, the outlet 20 of the second reservoir member 14 is shown in engagement with the tube 30. Specifically, the outlet 20 is fit inside the tube 30 to frictionally engage the second reservoir member 14 and the tube 30 and provide fluid communication between the chamber 16 and the lumen 71 of the tube 30. Because the tube 30 is flexible, it can deform to engage the outlet 20.

When the lumen 42, channel 40, chamber 16 and lumen 71 are interconnected, a passageway 72 from the fluid source 22 to the nozzle 28 (both shown in FIG. 1) is created. Further, when the reservoir members 12, 14 are connected, as in FIG. 2, the chamber 16 formed is able to hold medicaments. Structurally, the volume of the chamber 16 increases diametrically from the inlet 18 toward the distal end 66 of the first reservoir member 12. Then, the volume of the chamber 16 decreases diametrically from the proximal end 70 of the second reservoir member 14 to the outlet 20. In FIG. 2, shown positioned in the chamber 16 is a medicament 74, specifically, a selected anti-microbial agent. For the present invention, the anti-microbial agents 74 may be in different physical forms, including pills, capsules, gels, and powders, and may be bacteriocidal or bacteriostatic. More specifically, the anti-microbial agents 74 may include metallic salts (sodium chloride, bicarbonate or soda, povidone iodine, sodium hypochlorite, or other anti-microbial agents effective against Porphyromonas gingivalis, Prevotella intermedia, Bacteroides forsythus, Fusobacterium, Selenomonas, Centipeda periodontii, Spirochetes, Peptostreptococcus micros, Eubacterium, Actinobacillus actinomycetemcomitans, Eikenella corrodens, Capnocytophaga, Campylobacter rectus, Enteric rods/pseudomonads, Staphylococcus, Enterococcus faecalis, Candida, Protozoans (Amoebae and Trichomonads), and viruses. As further shown in FIG. 2, a filter 76 is positioned adjacent to the outlet 20 to prevent the medicament 74 from blocking the outlet 20. Also, in order to seal the components of the device 10, each interface between device components may be provided with resilient O-rings 78.

For purposes of the present invention, the first reservoir member 12 and the second reservoir member 14 are selected from a plurality of reservoir members 12 and 14. For instance, a small chamber 16 formed by a first reservoir member 12 and a second reservoir member 14 may have an axial length of 1¾ inches (1.75 in.) and a maximum diameter of ⅝ inches (0.616 in.). Further, a medium chamber 16 may have an axial length of 2⅜ inches (2.745 in.) and a maximum diameter of 1 inch. Also, a large chamber 16 may have a length of 2⅛ inches (2.116) and a maximum diameter of 1¼ inches (1.30 in.). For each reservoir member 12, 14, the diameter of the inlet 18 and the diameter of the outlet 20 may be selectively varied as well, though the diameter of the inlet 18 is preferably ¼ inch (0.30 in.) and the diameter of the outlet 20 is preferably 3/24 inches (0.2675 in.). Further, for each reservoir member 12, 14, the length of the outlet 20 may be selectively varied.

With the provision of reservoir members 12, 14 having chambers 16, inlets 18 and outlets 20 (and tubes) of varying diameters and lengths, a range of flow rates through the passageway 72 can be provided. For instance, given a flow rate from the fluid source 22, the selected length and diameters of the inlet 18, reservoir members 12, 14, and outlet 20 can determine a maximum flow rate through the passageway 72. As a result, the device 10 provides for proper treatment of selected tissue 34 with any desired medicament 74. For instance, proper treatment with metallic salts, iodine, etc. may require the application of these various agents over a period of time no longer than five minutes and at appropriate concentrations. After the dissolving rate of the topical agents is determined, and with the volume of the medicament 74, the maximum concentration of the medicament 74, and the period of application for the medicament 74 known, reservoir members 12, 14 having a properly dimensioned chamber 16, inlet 18 and outlet 20 may be selected. In this manner, the device 10 provides for the proper application of a wide range of medicaments 74.

In operation, the micro-organisms are identified, and a treatment plan is identified and implemented. Specifically, the medicament 74 is identified, the amount of medicament 74 is chosen, and the amount of time for the application of the medicament 74 is selected. As a result, an optimal flow rate for the fluid 32 is ascertained. In view of these determinations, the appropriate first reservoir member 12 and second reservoir member 14 are selected. After the components of the device 10 are interconnected, and the medicament 74 is positioned in the chamber 16, the water source 22 is activated to flow the water 32 through the passageway 72. The valve 26 and fluid source 22 may be manipulated to achieve the desired flow rate in view of the selected reservoir members 12, 14. As the water 32 passes the medicament 74, it dissolves or otherwise picks up some of the medicament 74 and carries the medicament 74 out of the nozzle 28 to irrigate the selected tissue 34. After the treatment is performed, the water source 22 is turned off. Then another medicament 74 may be positioned in the chamber 16 of the same reservoir members 12, 14, or in the chamber 16 of different sized reservoir members 12, 14, and the device components reconnected. When the device 10 is ready, the water source 22 is again activated and the water is adjusted to a desired flow rate. This process may be repeated for multiple medicaments 74, as desired. While FIG. 1 illustrates the use of the device 10 on oral tissue 34, the device 10 may be used on any type of tissue infected with micro-organisms.

While the particular Dental/Medical Oral Irrigation System as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for delivering a medicament to a selected tissue comprising the steps of:
Identifying the medicament to be delivered to the selected tissue;
choosing an amount of the medicament to be delivered to the selected tissue;
determining an amount of time for delivery of the chosen amount of medicament to the selected tissue;
connecting a first reservoir member to a fluid source;
engaging a second reservoir member to the first reservoir member to define a chamber, with the amount of medicament positioned in the chamber, and with the second reservoir member having an outlet;
attaching a tube to the outlet of the second reservoir, with said first reservoir member, second reservoir member and tube defining a passageway, and with said tube having a nozzle;
ascertaining an optimal flow rate for fluid through the passageway to deliver the chosen amount of the medicament to the selected tissue in the determined amount of time; and
establishing the optimal flow rate for fluid through the passageway to direct the fluid through the passageway and deliver a selected amount of the medicament from the passageway through the nozzle to the selected tissue in a predetermined amount of time.

2. A method as recited in claim 1 wherein the medicament comprises an initially-delivered medicament selected from the group consisting of chloramine T, saline, metallic salts, iodine, CHX, and therasol, and a later-delivered medicament comprising a topical agent.

3. A method as recited in claim 1 wherein the medicament is effective against at least one pathogen selected from the group consisting of Porphyromonas gingivalis, Prevotella intermedia, Bacteroides forsythus, Fusobacterium, Selenomoas, Sentipeda periodontii, Spirochetes, Septostreptococcus micros, Eubacterium, Actinobacillus actinomycetemcomitans, Eikenella corrodens, Capnocytophaga, Campylobacter rectus, Enteric rods/pseudomonads, Staphylococcus, Enterococcus faecalis, Candida, and viruses.

4. A method as recited in claim 1 wherein the medicament is an anti-microbial agent.

5. A method as recited in claim 1 wherein, during the ascertaining step, a concentration of the medicament in the fluid passing out of the nozzle is selected.

6. A method as recited in claim 1 wherein the tissue is oral tissue.

7. A method as recited in claim 1 wherein the medicament is in solid form when positioned in the chamber, and wherein the fluid dissolves the medicament when passing through the chamber.

8. A method as recited in claim 1 wherein the fluid is water.

9. A device for delivering a medicament to selected tissue comprising:
a first reservoir member for connecting to a fluid source;
a second reservoir member for engaging the first reservoir member to define a chamber, with the medicament positioned in the chamber, and with the second reservoir member having an outlet;
a tube attached to the outlet of the second reservoir, with said tube having a nozzle, wherein said first reservoir member, said second reservoir member and said tube define a passageway from the fluid source to the nozzle; and
a means for establishing an optimal flow rate for fluid through the passageway to deliver a chosen amount of the medicament through the nozzle to the selected tissue in a pre-determined amount of time.

10. A device as recited in claim 9 wherein the establishing means selects a concentration of the medicament in the fluid passing out of the nozzle.

11. A device as recited in claim 9 further comprising a filter positioned in the second reservoir member to prevent the medicament from blocking the outlet of the second reservoir.

12. A device as recited in claim 9 wherein the medicament is an anti-microbial agent and the formulation thereof is selected from a group consisting of a solid, a gel, and a powder.

13. A device as recited in claim 9 wherein the selected tissue is oral tissue.

14. A device as recited in claim 9 further comprising a plurality of differently sized first reservoir members and a plurality of differently sized second reservoir members, with said first reservoir member and said second reservoir member being selected from the respective plurality.

15. A method for delivering an anti-microbial agent to a selected oral tissue comprising the steps of:
choosing an amount of the anti-microbial agent to be delivered to the selected oral tissue;
determining an amount of time for delivery of the chosen amount of anti-microbial agent to the selected oral tissue;
providing a first reservoir member in fluid communication with a water source;
engaging a second reservoir member to the first reservoir member to define a chamber, with the chosen amount of the anti-microbial agent positioned in the chamber, and with the second reservoir member having an outlet;
attaching a tube to the outlet of the second reservoir, with said first reservoir member, second reservoir member and tube defining a passageway, and with said tube having a nozzle; and
ascertaining and establishing an optimal flow rate for water through the passageway to deliver the chosen amount of the anti-microbial agent to the selected oral tissue in the determined amount of time.

16. A method as recited in claim 15 wherein the antimicrobial agent is effective against at least one pathogen selected from the group consisting of Porphyromonos gingivalis, Prevotella intermedia, Bacteroides forsythus, Fusobacterium, Selenomonas, Centipeda periodontii, Spirochetes, Peptostreptococcus micros, Eubacterium, Actinobacillus actinomycetemcomitans, Eikenella corrodens, Capnocytophaga, Campylobacter rectus, Enteric rods/pseudomonads, Staphylococcus, Enterococcus faecalis, Candida, and viruses.

17. A method as recited in claim 15 wherein, during the ascertaining and establishing step, a concentration of the anti-microbial agent in the water passing out of the nozzle is selected.

18. A method as recited in claim 16 further comprising the step of varying the flow rate of the water through the passageway.

19. A method as recited in claim 15 wherein the anti-microbial agent is in solid form when positioned in the chamber, and wherein the water dissolves the anti-microbial agent when passing through the chamber.

20. A method as recited in claim 15 further comprising the step of preventing the anti-microbial agent from blocking the outlet of the second reservoir.
